(19) United States
(12) Patent Application Publication
(10) Pub. No.: US 2014/0276534 A1
(43) Pub. Date: Sep. 18, 2014

(54) NEEDLELESS INJECTOR

(71) Applicant: Nova Pneumatics LLC, Carthage, IL (US)

(72) Inventor: Michael Wood, Carthage, IL (US)

(73) Assignee: Nova Pneumatics LLC, Carthage, IL (US)

(21) Appl. No.: 13/827,983

(22) Filed: Mar. 14, 2013

(51) Int. Cl. A61M 5/30

(52) U.S. Cl. CPC A61M 5/30
USPC 604/500; 604/70

(57) ABSTRACT

A needleless injector includes an injector body having first and second opposite ends. There is a first piston disposed within the body. A second piston may also be disposed within the body configured with a second end for housing a nozzle. There is a medicine inlet for receiving medicine and a pressurized fluid inlet for receiving pressurized fluid. There is also a trigger for releasing the pressurized fluid to drive the first piston thereby driving the medicine through the nozzle for needleless delivery.

### FIELD OF THE INVENTION

The present invention relates to a needleless injector, method and system.

### BACKGROUND OF THE INVENTION

Vaccination or administration of medicine by injection has long been performed by using syringes with needles. However, there are problems with this method. Needles can cause damage which can be problematic as it may adversely affect the grading of an animal and result in financial loss for meat products. Moreover, use of needles risks needles breaking in the animal which can result in significant food safety issues.

Although it is known to use a needleless injector, various problems exist with various approaches to needleless injection. One problem with some needleless delivery system is the use of compressed air. Maintaining tanks can be expensive and creates delivery issues.

A still further problem relates to systems where medicine is maintained under pressure. Maintaining medicine under pressure can have adverse effects on the medicine.

What is needed is an improved needleless injector which addresses these and/or other issues.

### SUMMARY OF THE INVENTION

Therefore, it is a primary object, feature, or advantage of the present invention to improve over the state of the art.

It is a further object, feature, or advantage of the present invention to provide a needleless injector which allows for a multiple phase injection and delivery pressure profile.

It is another object, feature, or advantage of the present invention to provide a needleless injector which allows for multiple shots to be administered.

A still further object, feature, or advantage of the present invention is to provide a needleless injector which may be safely operated.

Another object, feature, or advantage of the present invention is to provide a needleless injector with an easy to adjust dosage.

Yet another object, feature, or advantage of the present invention is to improve food safety.

Another object, feature, or advantage of the present invention is to provide a needless injector with multiple stages.

A further object, feature, or advantage of the present invention is to provide a needleless injector which may be hydraulically or pneumatically driven.

A still further object, feature, or advantage of the present invention is to provide a needless injector that has a fully adjustable pressure profile for controlling injection and delivery of a medicine.

According to one aspect, the invention is a needleless injector. The needleless injector includes an injector body having first and second opposite ends. A first piston is disposed within the body, and a second piston may also be disposed within the body. The second end of the body includes a nozzle. A medicine inlet is provided for receiving medicine within the body. A pressurized fluid inlet is also provided for receiving pressurized fluid within the body. A trigger is operably configured for releasing the pressurized fluid to drive the first piston thereby driving the second piston and thereby driving the medicine through the nozzle for needleless delivery. In this aspect of the invention, the second piston may also be configured with a valve for controlling a pressure profile between the first and second valves.

According to another aspect, the invention is a needleless injector that includes an injector body having first and second opposite ends. A first piston is disposed within the body that includes a nozzle at a second end. The body also includes a medicine inlet for receiving medicine within a control volume between the first piston and the nozzle and a pressurized fluid inlet for receiving pressurized fluid. A trigger is configured for releasing the pressurized fluid to drive the first piston to a triggered position thereby driving the medicine from the control volume through the nozzle for needleless delivery. A valve may be disposed in the control volume for selectively controlling an injection pressure profile for the medicine. The dispensing pressure profile in the control volume may be a collapsed injection pressure profile for delivering the medicine.

According to another aspect, the invention is a method for needleless injection. The method includes providing a needleless injector with (a) an injector body having first and second opposite ends, (b) a first piston disposed within the body, (c) a nozzle at a second end of the body, (d) a medicine inlet for receiving medicine within a volume within the body, (e) a pressurized fluid inlet for receiving pressurized fluid, and (f) a trigger for releasing the pressurized fluid to drive the first piston. Exemplary steps of the method also include generating an injection pressure profile within the volume and collapsing the injection pressure profile to create a dispensing pressure profile. One or more pistons may be driven to collapse the injection pressure profile for delivering medicine through the nozzle during the collapsing step. By triggering the trigger the pressurized fluid is released.

One or more of these and/or other objects, features, or advantages of the present invention will become apparent from the specification and claims that follow. Different embodiments may have different objects, features, or advantages.

### BRIEF DESCRIPTION OF THE DRAWINGS

FIG. 1 is a block diagram illustrating an exemplary needleless injector system of the invention.

FIG. 2 is a block diagram illustrating another exemplary needleless injector system of the invention.

FIG. 3 is a pictorial representation of an exemplary configuration of a needleless injector of the invention.

FIG. 4 is a pictorial representation of another exemplary configuration of a needleless injector of the invention.

FIG. 5 illustrates a pressure profile for an injection.

### DETAILED DESCRIPTION

A needleless injector is provided. The needless injector is suitable for various types of uses including needleless injection of animals such as hogs, chickens, turkeys, cattle, and other types of livestock.

FIG. 1 provides a pictorial representation of a system 300 configured according to an exemplary aspect of the present invention. The system includes a needleless injector 334. The needleless injector 334 includes a control volume 316, a pre-delivery pressure profile 318, a controller 324, and a post-delivery pressure profile 326 for delivery 332 of injecting fluid 314. The control volume 316 within the needleless injector 334 is configured to receive a dosage of injecting fluid 314. The injecting fluid 314 within the control volume 316 has a pre-delivery pressure profile 318. The pre-delivery pressure profile 318 is controlled, for example, by one or more inputs to the control volume 316. In one aspect of the invention, input 310 changes the volume of the control volume 316 and input 312 changes the pressure within the control volume 316. One or more pistons may be operated to change the volume 310 in the control volume 316. For example, by decreasing the volume of the control volume 310 results in a change in pressure 312 within the control volume 316. Conversely, by increasing the volume within the control volume 316 results in a decrease in the in the pressure 312 in the control volume 316. Thus, the control volume 316, by change of the volume 310, may be configured to compress the injecting fluid 314 or expand the injecting fluid 314.

The pressure input into the control volume 316 may also be increased to compress the volume by way of a pressure change 312 resulting in a pressure profile within the control volume 316 or expand the volume by way of a pressure change 312 resulting in a vacuum profile within the control volume 316. The invention contemplates numerous variations for changing the pressure profile within the control volume 316. For example, any number of valves, pistons, cylinders, poppets, seats, seals, or like assemblies, devices or components may be configured within the control volume 316 to provide a change in volume 310 or change in pressure 312 resulting in a desired pressure profile for the delivery 332 of fluid 314 within the control volume 316.

The pressure profile within the control volume 316 may be controlled to provide a desired delivery pressure profile 318 and a post-delivery pressure profile 326. The needleless injector 334 may include a controller 324 having one or more inputs for providing one or more control operations for affecting the delivery pressure profile 318 and the post-delivery pressure profile 326. The controller 324 may be configured for delivery 332 of injecting fluid 314 by effectively managing controller 324 parameters which result in a differentiation between the post-delivery pressure profile 326 and the delivery pressure profile 318. The controller 324 may be configured within the control volume 316 for selectively opening and closing delivery 332 of injecting fluid 314 from the control volume 316. The control volume 316 may have the delivery pressure profile 318 prior to delivery 332 of injecting fluid 314 and the post-delivery pressure profile 326 after delivery 332 of injecting fluid 314. The controller 324 is preferably configured to manage the pre- and post-pressure profiles of the injecting fluid 314 upon delivery 332. By mechanical activation 328, electronic activation 330, pressure activation 322, and closing bias 320, controller 324 may be configured to optimize the difference between the post-delivery pressure profile 326 and the delivery pressure profile 318. In one embodiment, the closing bias 320 of a selectively closeable opening (e.g., valve) may be controlled to create a desired delivery pressure profile 318 and a desired post-delivery pressure profile 326. The closing bias 320 of the controller 324 may be controlled by pressure activation 322, mechanical activation 328 or electronic activation 330. By pressure activation 322 an unbalanced force acting on closing bias 320 may be configured to open and close the control volume 316 for providing delivery 332 of injecting fluid 314 while maintaining the injecting fluid 314 at a desired delivery pressure profile 318 and a desired post-delivery pressure profile 326. Mechanical activation 328 of the closing bias 320 may be configured as a spring biased against a poppet for selectively opening and closing and controlling the delivery pressure profile 318 and post-delivery pressure profile 326 of injecting fluid 314 housed within the control volume 316 and at the time of delivery 332. By electronic activation 330, the closing bias 322 within controller 324 may be optimized to manage the change in volume 310 or change in pressure 312 in the control volume 316 for delivery 332 of injecting fluid 314 at a desired delivery pressure profile 318 and post-delivery pressure profile 326. In one embodiment, an electronically controlled valve may be managed by controller 324 for providing the injecting fluid 314 at delivery 332 having the delivery pressure profile 318 and the post-delivery pressure profile 326. An example of a delivery pressure profile and the post-delivery pressure profile are shown in FIG. 5 and described in further detail below.

FIG. 2 is a pictorial representation of a system 400 configured for needleless injection of an injecting fluid 428. System 400 is but one configuration for delivery 426 of injecting fluid 428 in a needleless manner. System 400 includes a set of injecting profile parameters 416 for operatively controlling injecting profile control system 430 for providing delivery 426 of injecting fluid 428. The injecting profile control system 430 is configured to control the pressure profile of delivery 426 of injecting fluid 428. The injecting profile parameters 416 include for example, pressure 410, firing & exhaust 412, and expansion 414 to name a few. Each may be optimized and managed to provide support for operation of the injecting profile control system 430. The injecting profile control system 430 includes a control volume 422 actuatably controlled for delivery 426 of injecting fluid 428. A controller (not shown) may be configured in operable communication with injecting profile control system 430 for optimizing, managing and actuatably controlling control volume 422.

The control volume 422 is managed by the injecting profile control system 430 to have a delivery pressure profile 420 and a post-delivery pressure profile 424. The injecting profile control system 430 may be configured to manage the control volume 422 to optimize the timing at which the control volume 422 changes from the delivery pressure profile 420 to the post-delivery pressure profile 424 during the delivery 426 of injecting fluid 428. Optimization of the delivery pressure profile 420 and post-delivery pressure 424 in the control volume 422 may be controlled by injecting profile parameters 416. One of the injecting profile parameters 416 may include a pressure 410 parameter operably controlled, for example, by a pressure regulator assembly configured in operable communication with the injecting profile control system 430. Delivery of a desired pressure 410 from injecting profile parameters 416 to the injecting profile control system 430 is one aspect of the invention for regulating the pressure within the control volume 422 to have a desired delivery pressure profile 420 and a desired post-delivery pressure profile 424 during delivery 426 of injecting fluid 428. Expansion of pressure 410 or collapsing pressure 410 may be used to manage the pressure within the control volume 422 so that delivery 426 of injecting fluid 428 is governed by a delivery pressure profile 420 and after delivery a post-delivery pressure profile 424 resulting from actuation of pressure 410 of injecting profile parameters 416 acting on the control volume 422.

Pressure 410 may operate in conjunction with exhaust 412 and expansion 414 parameters. For example, in combination with the pressure 410 parameter, a firing & exhaust 412 parameter may be operably configured to control one or more aspects of the injecting profile control system 430. In an exemplary configuration of the present invention, firing & exhaust 412 parameter may include a firing or exhaust valve assembly. The pressure 410 during firing of delivery 426 of injecting fluid 428 may be controlled by injecting profile control system 430 for controlling the delivery pressure profile 420 and the exhaust 412 of pressure from the control volume 422 may also be controlled by injecting profile control system 430 for controlling the post-delivery pressure profile 424. An expansion 414 parameter may also be configured as part of the injecting profile parameters 416 for controlling delivery of a set of desired parameters from the injecting profile parameters 416 to the injecting profile control system 430. An expansion parameter 414 of the pressure 410 may be controlled and delivered to the injecting profile control system 430 for managing the post-delivery pressure profile 424 upon delivery 426 of injecting fluid 428. The injecting profile parameters 416 may be included as a system or subsystem of the needleless injector 418.

In one exemplary configuration, the injecting profile parameters 416 and injecting profile control system 430 are configured as a needleless injector 418. Operation of the needleless injector 418 may be supported in part by connecting injecting fluid 428 to delivery 426 managed by the injecting profile control system 430. Operation of the injecting profile parameters 416, namely the pressure 410, firing and exhaust 412 and expansion 414 may be managed, optimized and executed by a controller (not shown) or other like devices. The timing of operation of the injecting profile parameters 416 may also be controlled, at least in part, by a controller (not shown) or a like device. Similarly, injecting profile control system 430 may include one or more control operations or control devices for managing the injecting profile parameters 416.

FIG. 3 provides a pictorial representation of an exemplary configuration for a needleless injector 500 of the present invention. The needleless injector 500 includes an injector body 510 for carrying one or more of the components of the needleless injector 500. One component operably configured to be carried by the injector body 510 is a cylinder 512. One or more seals (e.g., O-rings) may be configured around the outer perimeter of the medicine piston 536 for seating against the interior wall of the injector body 510 for creating seal between the cylinder 512 wall and the medicine piston 536. A valve, such as a check valve 518 is operably configured at an inlet 520 passing through the wall of the injector body 510 into the cylinder 512. Similarly, an inlet 514 is configured to pass through the wall of the medicine piston 536. Both inlets 520 and 514 are configured to place the check valve 518 in communication with the medicine chamber 530 within the medicine piston 536. Medicine 516 is introduced into the medicine chamber 530 through the check valve 518, inlet 520 and inlet 514. The medicine piston 536 has a medicine cylinder 528 with a first end and a second opposite end terminating in a delivery cylinder 532 and nozzle 538 with a nozzle opening 534.

Also included within the medicine piston 536 is a piston 524 housing a valve 526. The present invention contemplates that valve 526 may be configured as a separate component from piston 524 and housed within the medicine chamber 530 of medicine piston 536. Both pistons 522 and piston 524 are configured within the medicine cylinder to seat against the inner surface of the medicine chamber at the medicine cylinder 528 wall via a seal such as an O-ring. Piston 522 and 524 are configured to move within the medicine cylinder 528 for changing the volume of the medicine chamber 530. The valve 526 may be configured as a poppet valve, electrically operated valve, pneumatically operated valve, hydraulically operated valve, or with a like operated valve for allowing passage of medicine 516 through piston 524. Valve 526 is configured, according to a preferred aspect of the present invention, to control the pressure profile of medicine 516 within medicine chamber 530 and the pressure profile of medicine 516 within delivery cylinder 532.

Upon introduction of medicine 516 into the medicine chamber 530 of the medicine piston 536, the medicine 516 is carried in the medicine chamber 530 by the medicine piston 536. The medicine 516, may for example, reside within medicine chamber 530 at atmospheric pressure or at a pressure above atmospheric pressure, but preferably at a pressure that approximates atmospheric pressure. By actuation of piston 522, the pressure profile for the medicine 516 within the medicine chamber 530 may be controlled before, during and after delivery of medicine 516 through delivery cylinder 532 and nozzle opening 534. Prior to firing the needleless injector 500, the medicine piston 536 may be moved within cylinder 512 of the injector body 510 to an injecting position, such as where nozzle 538 extends outside of the injector body 510 to present the nozzle opening 534 at a position capable of being placed in contact with an injecting surface. Movement of the medicine piston 536 within cylinder 512 of injector body 510 may occur simultaneously or sequentially with movement of piston 522 within medicine cylinder 528. Actuation of the medicine piston 536 and piston 522 may be pneumatically actuated, hydraulically actuated, electronically actuated, mechanically actuated or actuated using any like means. According to one aspect of the invention, cylinder 512 may be connected in communication with a source of pneumatically compressed air or hydraulically compressed fluid for actuating medicine piston 536 and advancing it forward within cylinder 512 of the injector body 510. One connection type includes pneumatically coupling to a pressurized pneumatic source. Alternatively, a connection could include hydraulically coupling to a pressurized hydraulic source. The piston 522 may be advanced forward within the medicine cylinder 528 in a similar manner. Advancing piston 522 within medicine cylinder 528 increases the pressure on the medicine 516 within medicine chamber 530, which in turn advances piston 524 forward toward the delivery cylinder 532. Piston 524 may be configured to stop at a desired position associated with a desired volume for delivery cylinder 532.

According to one embodiment, piston 524 is advanced toward the delivery cylinder 532 until it reaches a stopping point, for example, against the delivery cylinder 532. As piston 522 is advanced forward through the medicine cylinder 528, medicine 516 is compressed within the medicine chamber 530 to a desired pressure profile based upon the configuration and setting of valve 526. By way of example, valve 526 may be configured with a biasing element which is electronically, pneumatically, hydraulically, or mechanically biased to stay closed until the pressure within the medicine chamber 530 reaches a desired pressure profile for the medicine 516. In one embodiment, piston 522 may include a pin for actuating valve 526 at a desired position of piston 522 within medicine cylinder 528. Thus, for example, piston 522 may advance within medicine cylinder 528 until the forward movement of piston 522 mechanically actuates valve 526 thereby opening and releasing medicine 516 from within medicine chamber 530 through delivery cylinder 532 and nozzle opening 534. In another embodiment, a pressure sensor may be configured within chamber 530 for monitoring the pressure of medicine 516 as piston 522 advances forward within medicine cylinder 528. The pressure sensor may be configured in operable communication with an electronic control or solenoid for electronically controlling valve 526 and opening the valve when the pressure of medicine 516 within chamber 530 reaches a desired pressure profile. In another embodiment, a biasing element such as a spring may be operably connected to valve 526 for resistively closing valve 526 and opening to release fluid 516 from within chamber 530 at a desired pressure profile. In yet another embodiment, valve 526 may be hydraulically actuated and configured with a hydraulic release set point that is actuated to an open position upon the medicine 516 within medicine chamber 530 reaching a desired pressure profile. As previously indicated, for the afore-described embodiments, a source of pressurized hydraulic fluid or a source of pressurized pneumatic air may be configured in operable communication with the needleless injector 500 for supporting operation of the various configurations of valve 526.

Upon actuation of valve 526, medicine 516 within medicine chamber 530 advances forward through piston 524 into the delivery cylinder 532 connected in fluid communication with nozzle opening 534. The medicine 516 is advanced through the delivery cylinder 532 and nozzle opening 534 having the pressure profile set up within medicine chamber 530. In one embodiment of the invention, the pressure profile within medicine chamber 530 is raised to a desired pressure by advancement of piston 522, such as a pressure suitable for injecting medicine 516 through the skin of the patient or recipient. As the pressure profile within the medicine chamber 530 expands upon advancement of medicine 516 through valve 526 and into the patient or recipient, the back pressure on the dosage of medicine 516 being administered decreases immediately so that the medicine is delivered under a collapsing or collapsed pressure profile having a pressure less than the initial pressure within medicine chamber 530 used to pierce the skin of the patient or recipient. The medicine piston 536 may be configured so that the pressure profile collapses within the medicine chamber 530 and within the delivery cylinder 532 simultaneously or sequentially. For example, the collapsing of the pressure field within these two respective locations may be sequentially whereas, for example, the collapsing of the pressure field within the delivery cylinder 532 lags behind the collapsing of the pressure field within medicine chamber 530. An illustration of a pressure profile is shown in FIG. 5 and described below.

FIG. 4 is a pictorial representation of another configuration for a needleless injector 600 of the present invention. The needleless injector 600 includes an injector body 611 for housing one or more operational and functional components for providing medicine 616 at the nozzle opening 634. Housed within the injector body 611 is a medicine chamber 622. The medicine chamber includes a medicine inlet 614 connected in communication with a check valve 618 adapted to receive medicine 616 from a medicine source (not shown). The check valve 618 is to prevent backflow of medicine 616 in a direction opposite the arrow shown, or otherwise out of medicine chamber 622 through medicine inlet 614.

A piston 620 is also housed within medicine chamber 622 and includes one or more seals (e.g., O-rings) for sealing off the piston against the inner surface of the medicine chamber 622. The piston 620 is moveable within the medicine chamber 622. For example, the piston 620 may be advanced from a first position to a second position (e.g., moving from left to right) toward the nozzle 635. The piston 620 may be retracted from right to left back against the inlet 624 adjoining the pressure regulator chamber 668 and medicine chamber 622 by actuation of a vacuum at inlet 624 or pressure within medicine chamber 622.

A dosage of medicine 616 dispensed into the medicine chamber 622 is dispensed out nozzle 635 through nozzle opening 634 as piston 620 is advanced from left to right toward nozzle 635. The pressure profile acting on piston 620 for providing a desired pressure profile acting on medicine 616 during injection, dispensing and post-injection are further described below in FIG. 5. For controlling the pressure profile acting on the dispensing sequence, a pressure regulator chamber 668, firing and exhaust valve chamber 666, and expansion chamber 662 are housed, according to one embodiment of the invention, within injector body 611. By one or more conduits, passageways or channels, a fluid body may be communicated between one or more of the aforementioned chambers. The fluid body may include a hydraulic or pneumatic fluid body. Preferably, each chamber is connected in communication with medicine chamber 622 for actuating piston 620 from a neutral position to an actuated position for dispensing medicine 616 through nozzle opening 634 in nozzle 635. For example, for firing piston 620 within medicine chamber 622 a firing and exhaust valve chamber 666 is provided. The firing and exhaust valve chamber may include, amongst other things, one or more pistons 646 and 650 operatively sealed against the inner side walls of chamber 666 to allow movement of the piston within the chamber. An inlet 644 into the chamber 666 is provided in the injector body 611. Inlet 644 may be connected in communication with a hydraulic or pneumatic source for actuating piston 646. Piston 646 actuates valve 648 thereby opening a poppet 656 which is resistively biased by a resistive element 652. The resistive element 652 may include a spring, elastomer element, or any like resistive element. The resistive element 652 biases the poppet 656 against valve 648. In the open position, a fluid medium passes through opening 654, valve 648 and through opening 672 in the wall that separates the pressure regulator chamber 668 from the firing and exhaust valve chamber 666.

In operation, a fluid body is introduced through inlet 644 for actuating piston 646 to open valve 648 which is resistively biased by 652 to close poppet 656 upon terminating fluid body pressure at inlet 644. In the open position, a fluid body such as air is permitted to pass through opening 654 and valve 648 through opening 672 separating chambers 666 from chamber 668. The resistive element 652 may be configured to control the pressure at which poppet 656 opens allowing the fluid body to travel through valve 648. When the needleless injector 600 is fired an actuation process occurs in chamber 666 allowing air to pass into the pressure regulator chamber 668. The reverse is also true for providing an exhaust function using chamber 666. Upon actuation of piston 646, valve 648 opens allowing air to exhaust from opening 672 through valve 648 and back out opening 654 in the firing and exhaust valve chamber 666. Upon completion of the exhaust cycle the poppet 656 is biased back to a closed position by the resistive element 652 thereby closing valve 648 and terminating the passage of a fluid body, such as air, through opening 672 and out opening 654 for exhausting the fluid body from the needleless injector 600.

In addition to the firing and exhaust valve chamber 666, a pressure regulator chamber 668 may be provided for operably controlling the pressure profile acting on piston 620. The fluid body of air, pneumatic fluid or the like communicated through opening 672 from firing and exhaust valve chamber 666 enters the pressure regulator chamber 668 which is configured to regulate the pressure of the entering fluid body to a desired pressure profile for acting on piston 620. The pressure regulator chamber 668 may be configured to provide a desired pressure profile at inlet 624 into medicine chamber 622. For controlling or regulating the pressure within chamber 668 a plurality of pistons are movably enclosed within the chamber by a seal, such as an O-ring, between the piston and the interior wall of the chamber. For example, a piston 630 and piston 632 may be enclosed within the chamber 668. A valve 670 is also disposed with the chamber 668 between pistons 632 and piston 630. One or more openings 640 and 638 may be included in the wall of the injector body 611 for allowing the flow of a fluid body into and out of the pressure regulator chamber 668. A fluid body, such as air, is permitted to flow through the pressure regulator chamber 668 into the medicine chamber 622 for actuating piston 620 upon actuation of valve 670. Valve 670 includes a poppet 626 resistively biased by a resistive element 628 and a pin 636 operably connected to the poppet 626 for actuating the poppet 626 and the valve to 670 to an open position. Another resistive element 642 may be connected in operable configuration with the pin 636. The resistive elements 628 and 642 may be configured so that a greater pressure or lesser pressure is required at inlet 610 for actuating piston 632 and subsequently actuating pin 636 which opens valve 670. In the open position, a fluid body such as air is permitted to flow through one or more of the openings 640 and 638 through valve 670 and inlet 624 into the medicine chamber 622. The inlet 610 to chamber 668 may be connected in fluid communication with a source of pneumatic compressed air or a source of compressed hydraulic fluid. Thus, the pressure regulator chamber 668 and/or the firing and exhaust valve chamber 666 may be hydraulically coupled to a hydraulic pressure source or pneumatically coupled to a pneumatic pressure source.

According to a desirable configuration, resistive elements 642 and 628 may be configured so that more or less pressure is required for regulating the pressure acting on piston 620 for injecting medicine 616 into the skin of a recipient or patient via nozzle opening 634. By way of an example, if a higher pressure is desired, resistive element 628 may have a greater resistance than resistive element 642. Conversely, if a lower pressure profile is desired, resistive element 642 may be configured to have biasing effect equal to or marginally less than the biasing effect on valve 670 so that a smaller pressure is capable of opening valve 670. Resistive elements 642 and 628 may be mechanical resistive elements such as a spring, or other biasing type means. Other resistive elements may be used such as an electrical component that biases movement of pin 636 and poppet 626. A solenoid controlled by a controller may be used to resistively bias elements 642 and 628. Similarly, pneumatic and hydraulic resistive components may be configured in the place of resistive elements 642 and 628 to control movement of pin 636 and poppet 626 thereby opening and closing valve 670 according to a desired pressure profile for acting on piston 620.

As previously described, one aspect of the invention is to provide a high pressure profile upon injection of the medicine 616 and a collapsing pressure profile after the skin is broken and the medicine begins to inject. In combination or separately, the firing and exhaust valve chamber 666 and pressure regulator chamber 668 provide a desired pressure profile acting on piston 620 for breaking the skin of the recipient or patient with the medicine 616 injected via nozzle opening 624. Upon piercing the skin the pressure profile for dispensing the medicine 616 through nozzle 635 is changed according to aspects of the invention which have previously been described and are now described. One embodiment includes expanding collapsing pressure of the medicine 616 by an expansion chamber 662, which may be housed within injector body 611. The expansion chamber 662 includes an inlet 664 for receiving a fluid body through an inlet 658 in a wall of the injector body 611. A fluid body such as air from a pressurized source passes through inlet 658 into the expansion chamber 662. The inlet 664 is provided for allowing a fluid body to enter into the expansion valve 612. Upon entering the expansion valve 612 the fluid body expands and is allowed to pass through opening 660 in the injector body 611. According to one exemplary configuration of the needleless injector 600, opening 660 and expansion chamber 662 is connected in fluid communication with the medicine chamber 620. The pressure profile previously described acting on piston 620 may be expanded and collapsed by permitting such expansion of the pressure field via the expansion valve 612 which may be permitted to exhaust from the injector body 611 via opening 658. The expansion valve 612 may be configured to control the rate of expansion of the pressure profile or otherwise the rate at which the pressure profile collapses so that the piston 620 is actuated in a manner to continue delivering the medicine 616 through nozzle 635 at a desired pressure and rate of delivery, preferably a high pressure to pierce the skin and start initial deliver of medicine 616 followed by a collapsing pressure profile for delivering of a greater or remaining portion of the medicine 616.

Note that such an injector provides various advantages. One such advantage is that the medicine itself is not stored while under pressure thus problems associated with medicine under pressure are avoided. Another advantage is that a pump unit (not shown) may be hydrostatically coupled to the needless injector body. The pump unit may be driven with a standard hydraulic pump or may be driven pneumatically.

FIG. 5 illustrates one example of a pressure profile 120 for pressure as a function of time. As shown in FIG. 5, there is no pressure until the device is triggered at time 122, once triggered the pressure profile quickly builds. At time 124 the expansion process or collapsing of the pressure field (see, e.g., collapsed pressure field 126) occurs at a controlled rate for decreasing the pressure profile during delivery of the medicine. This allows for different pressures to be applied. For example, a first pressure which is a very high pressure may be used to break the skin of the animal or patient and then in the second stage a lower pressure may be used to deliver the medicine. This may be achieved through the apparatus, method and systems of the invention described above. Thus, for example, a valve such as the check valve may be used to control the outflow of the medicine causing pressure to expand and the pressure profile to collapse. When the medicine is released, then it is at a higher pressure at the nozzle which then drops off for the remainder of the medicine deliver.

The foregoing description has been presented for the purposes of illustration and description. It is not intended to be an exhaustive list or limit any of the disclosure to the precise forms disclosed. It is contemplated that other alternatives or exemplary aspects are considered included in the disclosure. The description is merely examples of embodiments. For example, the exact number of pistons and valves and there arrangement may be changed according to the type of fluid type used, the desired pressure profile, and the operational pressure and volumetric delivery of the injector. It is understood that any other modifications, substitutions, and/or additions may be made, which are within the intended spirit and scope of the disclosure. From the foregoing, it can be seen that the disclosure accomplishes at least all of the intended objectives.

What is claimed is:

1. A needleless injector, comprising:
an injector body having first and second opposite ends;
a first piston disposed within the body;
a second piston disposed within the body;
a nozzle at a second end of the body;
a medicine inlet for receiving medicine;
a pressurized fluid inlet for receiving pressurized fluid;
a trigger for releasing the pressurized fluid to drive the first piston thereby driving the second piston and thereby driving the medicine through the nozzle for needleless delivery.

2. The needleless injector of claim 1 further comprising a volume between the first and second piston having a pressure profile comprising an injection pressure for the medicine and a delivery pressure for the medicine.

3. The needleless injector of claim 2 wherein the delivery pressure comprises a collapsing pressure field within the volume.

4. The needleless injector of claim 1 wherein the second piston includes a valve for controlling a pressure profile between the first and second valves.

5. The needleless injector of claim 1 wherein the first piston has a actuated position in operable interaction with a biasing element on the second piston to control a medicine injection and delivery pressure profile.

6. The needleless injector of claim 1 further comprising a pump unit hydrostatically coupled to the pressurized fluid inlet.

7. A needleless injector, comprising:
an injector body having first and second opposite ends;
a first piston disposed within the body;
a nozzle at a second end of the body;
a medicine inlet for receiving medicine within a control volume between the first piston and the nozzle;
a pressurized fluid inlet for receiving pressurized fluid;
a trigger for releasing the pressurized fluid to drive the first piston to a triggered position driving the medicine from the control volume through the nozzle for needleless delivery;
a valve disposed in the control volume for selectively controlling an injection pressure profile for the medicine;
a dispensing pressure profile in the control volume comprising a collapsed injection pressure profile for delivering the medicine.

8. The needleless injector of claim 7 wherein the first piston includes means for actuating the valve to an open position.

9. The needleless injector of claim 7 further comprising a second piston disposed within the body, the valve carried by the second piston.

10. The needleless injector of claim 7 further comprising a second piston within the control volume and a pressure profile between the first and second pistons for actuating the valve to an open position for delivering the medicine.

11. The needleless injector of claim 7 further comprising a biasing element in operable connection with the valve and actuated by the first piston in the triggered position.

12. The needleless injector of claim 11 wherein the injector body is disposed within a cylinder having a trigger with a actuated position corresponding to the triggered position.

13. A method for needleless injection, the method comprising:
providing a needleless injector comprising (a) an injector body having first and second opposite ends, (b) a first piston disposed within the body, (c) a nozzle at a second end of the body, (d) a medicine inlet for receiving medicine within a volume within the body, (e) a pressurized fluid inlet for receiving pressurized fluid, and (f) a trigger for releasing the pressurized fluid to drive the first piston;
generating an injection pressure profile within the volume;
collapsing the injection pressure profile to create a dispensing pressure profile;
driving one or more pistons to collapse the injection pressure profile;
delivering medicine through the nozzle during the collapsing step; and
triggering the trigger to release the pressurized fluid.

14. The method of claim 13 further comprising collapsing the injection pressure profile by actuation of a second piston disposed within the body.

15. The method of claim 13 further comprising opening a valve disposed within the volume for collapsing the injection pressure profile by moving the first piston.

16. The method of claim 13 further comprising simultaneously moving a second piston disposed in the volume with the first piston for generating the injection pressure profile.

17. The method of claim 13 further comprising a second piston disposed within the body and separated from the first piston by the volume, controlling a change in the volume for:
a. generating the injection pressure profile for injecting the medicine; and
b. generating a delivery pressure profile for delivering the medicine.

18. The method of claim 13 further comprising biasing movement of the one or more pistons for controlling the injection pressure profile.

19. The method of claim 13 further comprising housing a valve within the one or more pistons for controlling a rate of collapse for the injection pressure profile.

20. The method of claim 13 further comprising hydrostatically coupling a pump unit to the pressurized fluid inlet.
